(19) United States
(12) Patent Application Publication
(10) Pub. No.: US 2023/0074922 A1
(43) Pub. Date: Mar. 9, 2023

(54) PROGRAMMABLE DEVICE FOR PATHOGEN ?POINT-OF-CARE? TESTING

(71) Applicant: CECIL FRED MOTLEY, Rolling Hills Estates, CA (US)

(72) Inventor: CECIL FRED MOTLEY, Rolling Hills Estates, CA (US)

(21) Appl. No.: 17/406,334

(22) Filed: Aug. 19, 2021

(51) Int. Cl. A61B 5/08; A61B 5/00

(52) U.S. Cl. CPC A61B 5/08; A61B 5/7267; A61B 5/0075; A61B 5/002; A61B 5/682; A61B 5/6819; A61B 5/0004; A61B 5/0071

(57) ABSTRACT

This invention is a programmable, mobile, and reusable point-of-care-testing (POCT) unit for identifying pathogens and their viability state in the respiratory airways realis tempus. The device can be used to test for COVID-19 infections in individuals entering or exiting venues (i.e. schools, restaurants, bars, sporting events, etc.). The POCT unit is capable of performing thousands of tests without maintenance or repair. The POCT unit employs fluoresced spectra analysis (FSA) to uniquely identify the specific bacteria or virus and their relative concentration level based on spectral pattern recognition. Additionally, the POCT unit identifies the living or dead state of bacteria or the active or inactive state of a virus. Automatic pattern recognition of the bacteria or virus spectrum is done using Artificial Intelligence (AI) Deep Learning Neural Networks (DLNN). The DLNN computational process is performed at a remote site linked to the POCT unit by a smartphone or lap-top online connection. The POCT unit is an “at patient” testing instrument for identifying pathogen including SARS-CoV2, SWINE-FLU, H1N1, E-BOLI, Influenza, etc. The POCT unit response time is driven by the SmartPhone connectivity time or the laptop computational ability. The identification of a specific pathogen is determined by the programming of the DLNN and therefore useable for identifying current and future respiratory bacterial or viral infections by adjusting the DLNN software using new training data. The POCT unit has three configurations, namely, a mobile unit connected by Smartphone or PC and a personal home user version connected through Bluetooth to a SmartPhone.

### PUBLICATION CLASSIFICATION

| | |
|---|---|
| A61K 9/00 | (2020.01) |
| A61M 16/04 | (2020.01) |
| A61M 11/00 | (2006.01) |
| A61M 15/00 | (2020.01) |
| A61K 9/0073 | (2020.01) |
| A61K 9/0078 | (2020.01) |
| A61K 9/008 | (2020.01) |

### REFERENCES CITED

### Patent Documents U.S.

| | | |
|---|---|---|
| 10,712,259 | 7/2020 | Jessen et al. |
| 10,714,209 | 7/2020 | Pastrana - Rios |
| 10,716,499 | 7/2020 | Freeman |
| 10,718,668 | 7/2020 | Gu et al |
| 10,746,600 | 8/2020 | Lambert |

### Other Publications

(¹) “The Use of UV Light to Reduce Infections Associated with Central Venous, Arterial, and Urinary Catheters”, Motley et al, CMS Publication, Jul. 16, 2015.
(²) “Evidence that the spectral dependence of light absorption by aerosols is affected by organic carbon” JOURNAL OF GEOPHYSICAL RESEARCH, 2004, Vol. 109.
(³) “UV Inactivation of Pathogenic and Indicator”, Chang et al, APPLIED AND ENVIRONMENTAL MICROBIOLOGY, June 1985, Vol. 49, No. 6, p. 1361-1365.
(⁴) “PATTERN RECOGNITION with NEURAL NETWORKS in C⁺⁺, Abhijit S. Pandya and Robert B. Macy, 1996, CRC Press, Inc., ISBN 0-8493-9462-7.
(⁵) Optical Diagnostic of Dengue Virus Infected Human Blood using Raman, Polarimetric and Fluorescence Spectroscopy, Shamaraz Firdous and Shahzad Anwar, Chapter 5.

### BACKGROUND OF THE INVENTION

### 1. Field of Invention

Millions of people are being tested for SARS-CoV-2 (COVID-19) around the world and the tests themselves, though a crucial starting point in efforts to flatten the spread of the virus have become an impediment. There exist an urgent need to identify bacteria and viruses in near real-time for the purpose of clinical treatment as well as infection tracking within the world's population. Currently, FDA approved pathogen test require hours to days to process with varying reliability and are not readily useable for large populations.

The Center for Disease Control and Prevention (CDC) current approved testing methods are listed in Table 1.0. Each of these methods requires swabs of nasal, sputum or endotracheal aspirate and is primarily used for influenza testing. Other conventional methods for virus characterization include virus isolation, immunostaining, and PCR sequencing.

Many tests are being investigated and a few have been issued emergency utilization authorization (EUA) due to the urgent need for rapid testing. These tests require some type of chemical process and each test kit is disposable. This invention is reusable and do not require a chemical process.

TABLE 1
Influenza Virus Testing Methods

| | Types | | | | CLIA |
|---|---|---|---|---|---|
| Method¹ | Detected | Acceptable Specimens² | Test Time | | Waived³ |
| Rapid Virus Diagnostic Tests (antigen | A and B | NP swab, aspirate or wash, nasal | <15 | min. | Yes/No |
| detection) | | swab, aspirate or wash, throat | | | |
| | | swab | | | |
| Rapid Molecular Assay [influenza | A and B | NP swab, nasal swab | 15-30 | minutes | Yes/No |
| viral RNA or nucleic acid detection] | | | | | |
| Immunofluorescence, Direct (DFA) | A and B | NP swab or wash, bronchial | 1-4 | hours | No |
| or Indirect (IFA) Florescent Antibody | | wash, nasal or endotracheal | | | |
| Staining [antigen detection] | | aspirate | | | |
| RT-PCR (singleplex and multiplex; | A and B | NP swab, throat swab, NP or | Varies (1 to 8 | | No |
| real-time and other RNA-based) and | | bronchial wash, nasal or | hours, varies | | |
| other molecular assay | | endotracheal aspirate, sputum | by the assay) | | |
| Rapid cell culture (shell vials; cell | A and B | NP swab, throat swab, NP or | 1-3 | days | No |
| mixtures; yields live virus) | | bronchial wash, nasal or | | | |
| | | endotracheal aspirate, sputum; | | | |
| | | (specimens placed in VTM) | | | |
| Viral tissue cell culture (conventional; | A and B | NP swab, throat swab, NP or | 3-10 | days | No |
| yields live virus) | | bronchial wash, nasal or | | | |
| | | endotracheal aspirate, sputum | | | |
| | | (specimens placed in VTM) | | | |

NP—nasopharyngeal,
VTM—viral transport media,
CLIA—Clinical Laboratory Improvement Amendments of 1988

### 2. Technical Background

This invention is based on the use of optical spectroscopy to identify the presence of bacteria or viruses in the respiratory airway. Optical diagnostic apparatus are very effective as compared to conventional disease detection methods. Optical techniques are noninvasive, direct, cost-effective, and easy to use with high specificity, sensitivity, and small size. Optical diseases diagnostic research and development has been useful to healthcare, environmental applications, biotechnology industry, and medical sciences.

This invention uses Raman Spectroscopy which occurs when monochromatic radiation is incident upon a sample and this light interacts with the sample in some fashion. It may be reflected, absorbed or scattered in some manner. It is the scattering of the radiation that is referred to as Raman Spectroscopy which identifies the sample's molecular structure. If the frequency (wavelength) of the scattered radiation is analyzed, not only is the incident radiation wavelength seen (Rayleigh scattering) but also, a small amount of that radiation is scattered at some different wavelength (Stokes and Anti-Stokes Raman scattering). Only 1×10⁻⁷ of the scattered light is Raman. It is the change in wavelength of the scattered photon which provides the chemical and structural information.

Each bacteria and virus has a unique Fluoresced Spectra Analysis (FSA) pattern. FIG. 1 illustrates an example application of Raman Spectroscopy, a form of FSA, for detection of the spectral pattern of dengue virus in blood. The FSA pattern for normal blood 101 is shown in FIG. 1 and the FSA pattern for blood with the Dengue virus 201 (Anwar⁵) is shown in FIG. 2. Clearly, the spectra patterns are different and the sensitivity is sufficient to uniquely identify the Dengue virus. Raman spectrometers are available from several manufacturers however, the FSA output is examined manually by scientist or healthcare professionals which can be a time consuming process. This invention uses Artificial Intelligence (AI) to identify the specific FSA patterns of subject bacteria and viruses as well as their pathogen colony level and viability state 301 shown in FIG. 3, thus providing a reusable mobile near real-time point-of-care infection test.

### 3. Background Art

Current patents that address detection and identification of bacteria, viruses, fungal, and microorganism using Raman Spectroscopy techniques include:

U.S. Pat. No. 10,746,600 This disclosure for a Raman probe has a scanning optical bench that enables a 1-cm linear traverse across a target rock or soil, both on target materials as encountered and on fresh surfaces of rocks exposed by abrasion or coring. From these spectra, it is possible to identify major, minor, and trace minerals, obtain their approximate relative proportions, and determine chemical features (e.g., Mg/Fe ratio) and rock textural features (e.g., mineral clusters, amygdular fill, and veins). It is also possible to detect and identify organic species, graphitic carbon, and water-bearing phases. Extensive performance tests have been done on a brassboard model of the MMRS using a variety of geological materials (minerals, rocks, Martian meteorites, etc.). This device does not provide an instrument for identifying microorganisms.

U.S. Pat. No. 10,714,209 This disclosure characterizes proteins, peptides, and/or peptoids via two-dimensional correlation spectroscopy and/or two-dimensional co-distribution spectroscopies. This technique does not provide an instrument for identifying microorganisms.

U.S. Pat. No. 10,712,259 This invention relates to a photoacoustic sensor system for detecting target molecules in air samples or compressed air samples. This technique does not provide an instrument for identifying microorganisms.

U.S. Pat. No. 10,716,499 This invention is for optical spectroscopic devices, apparatus, systems and methods useable for physiological monitoring from intraosseous, subosseous, epidural, subdural, intraventricular, intramuscular, sub-adipose and other subcutaneous locations. This technique does not provide an instrument for identifying microorganisms.

U.S. Pat. No. 10,718,668 This invention is for an advanced miniature optical spectrometer that is resistant to mechanical vibration. This technique does not provide an instrument for identifying microorganisms.

### SUMMARY OF INVENTION

This invention provides a reusable mobile point-of-care testing for bacterial and viral infections present in the respiratory airways. The point-of-care testing unit identifies the presence of MRSA, H1-N1, MERS, SARS, and COVID-19 type infections present at ACE-2 receptor sites in the oral airway. The identification process is based on the unique fluoresced spectra pattern of a particular microorganism. Deep Learning Neural Networks are used to automatically identify their unique spectral patterns. The execution of the neural networks is performed at a remote site connected to the mobile point-of-care testing unit by SmartPhone or PC. The test response time is a function of the SmartPhone or PC computation time and the remote site AI processing. Typical delays are less than 30 seconds. The remote site provides massive data base storage, super computers, and information processing required for individual tracking. The point-of-care test unit is completely reusable for thousands of test without the need for maintenance or repair.

### BRIEF DESCRIPTION OF THE DRAWINGS

The invention is illustrated by way of example and not by way of limitation in the figures of the accompanying drawings in which like references indicate similar elements. It should be noted that references to “an” or “one” embodiment in this disclosure are not necessarily to the same embodiment and such references mean at least one.

FIG. 1 illustrates the fluoresced spectrum of normal blood obtained from Raman Spectroscopy.

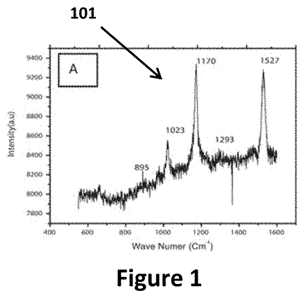

FIG. 2 illustrates the fluoresced spectrum of blood infected by the Dengue virus obtained from Raman Spectroscopy.

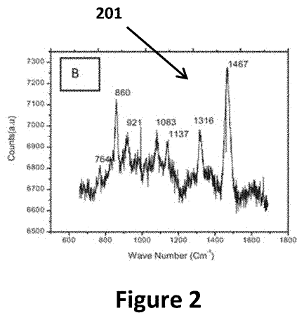

FIG. 3 illustrates the spectral relationship between living and dead E. coli.

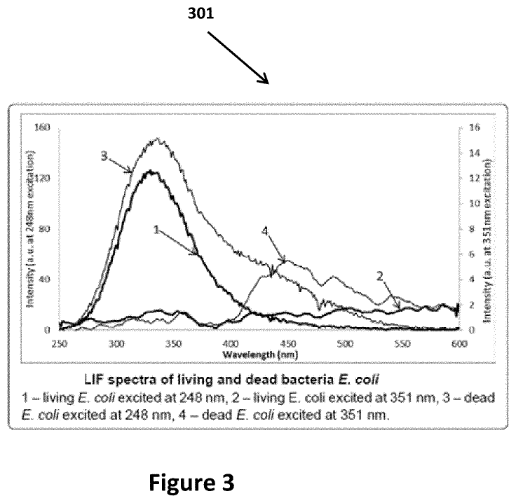

FIG. 4 illustrates the functional block diagram of the first embodiment of the invention.

FIG. 5 illustrates the functional block diagram of the second embodiment of the invention.

FIG. 6 illustrates the functional block diagram of the third embodiment of the invention.

FIG. 7 illustrates the probe inserted in the upper respiratory airway anatomical structures.

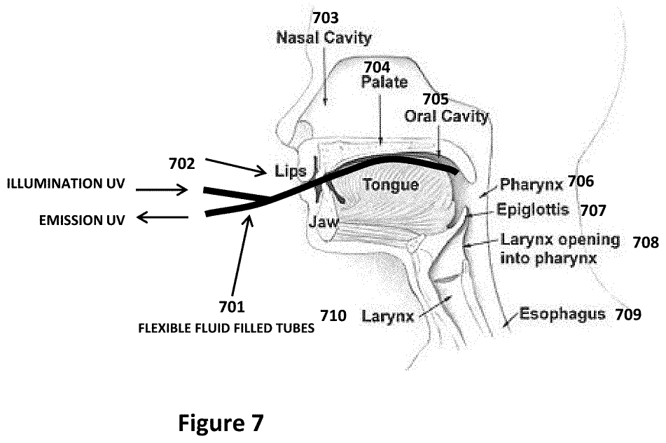

FIG. 8 illustrates the WAND inserted in the upper respiratory airway anatomical structures.

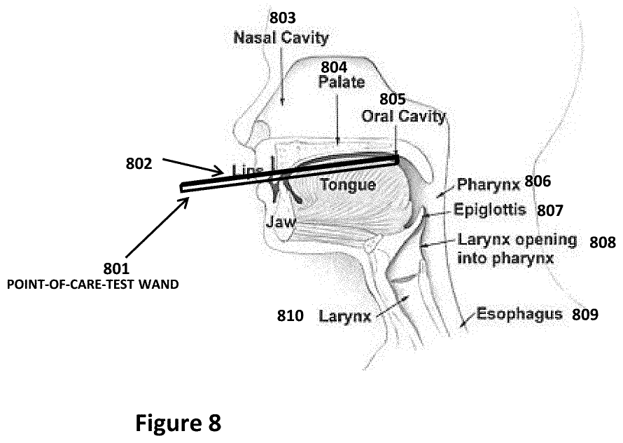

FIG. 9 illustrates the “Point-of-Care-Tester” Deep Learning Neural Network Architecture used to identify the specific bacteria or virus detected by the RAMAN Spectroscopy.

FIG. 10 illustrates the processing components of the remote AI Processing Center.

### DETAIL DESCRIPTION

The following detailed description and the accompanying drawings to which it refers are intended to describe some, but not necessarily all, examples or embodiments of the invention. The described embodiments are to be considered in all respects as illustrative and not restrictive. The contents of this detailed description and the accompanying drawings do not limit the scope of the invention in any way.

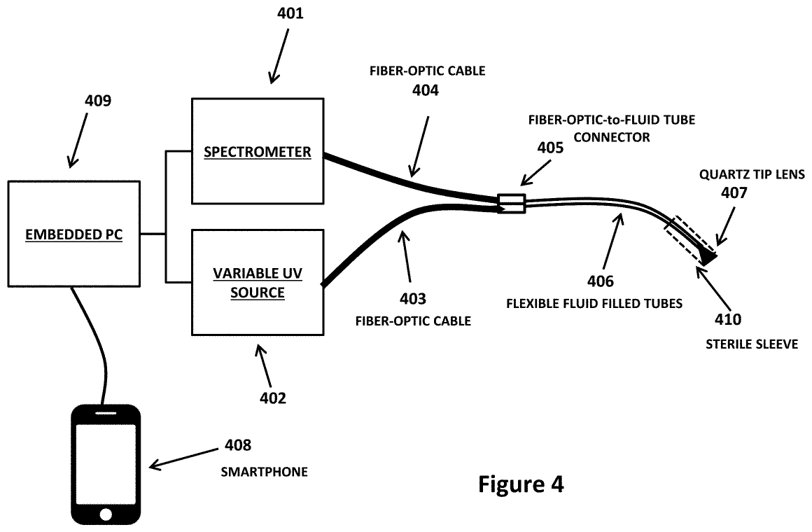

One embodiment of the POCT invention is illustrated in FIG. 4. This configuration uses a variable UV source 402 with a 200 to 1300 nm adjustable range in increments of 1 nm. This is the illumination light source that fluoresce the microorganism. A fiber optic link 403 transfers the UV light to a fiber-optic-to-fluid-tube converter 405. The dual fluid filled tube 406 output of the converter is very flexible and easily manipulated for insertion into the oral cavity for delivery of the illumination light and reception of the emitted light. The end of the flexible fluid tube pair is a dual quartz lens 407 forming a test probe for the POCT unit. Quartz material is used due to its low attenuation of light in the UVC and UVB bands. The second fluid filled flexible tube of the pair carries the emitted fluoresced light back to the converter 405 where it is delivered to the 300 to 4300 nm Raman Spectrometer 401 through a fiber optic link 404. A replaceable sterile sleeve 410 is placed over the distal end of the flexible fluid filled tube pair to allow reuse of the probe by multiple patients. The Raman Spectrometer extracts the spectra from the emitted light which is used to identify the infection microorganism and associated contamination level. An embedded PC 409 is included to interface to a SmartPhone 408 via Bluetooth or USB connection. The SmartPhone transfers the spectral data to a remote site where a supercomputer executes an AI DLNN trained to identify a specific bacterial or viral pattern and returns the test results.

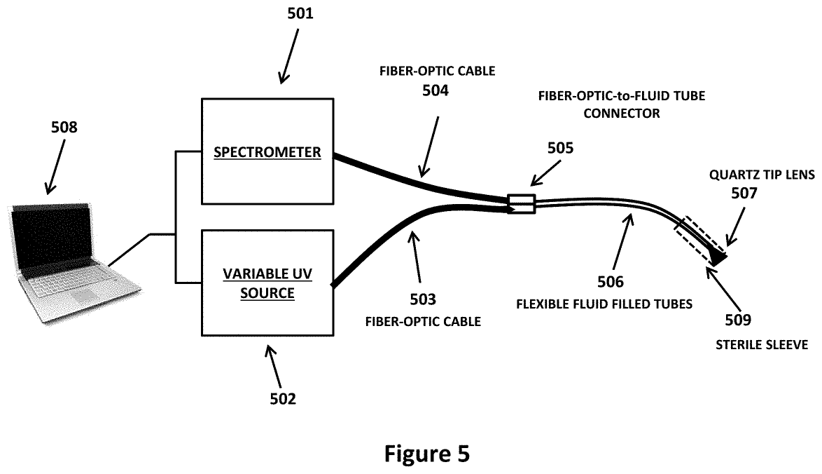

A second embodiment of the POCT invention is illustrated in FIG. 5. This configuration uses a variable UV source 502 with a 200 to 1300 nm adjustable range in increments of 1 nm. This is the illumination light source that fluoresce the microorganism. A fiber optic link 503 transfers the UV light to a fiber-optic-to-fluid-tube converter 505. The dual fluid filled tube 506 output of the converter is very flexible and easily manipulated for insertion into the oral cavity 701, 705 for delivery of the illumination light and reception of the emitted light. The end of the flexible fluid tube pair is a dual quartz lens 507 forming a test probe for the POCT unit. Quartz material is used due to its low attenuation of light in the UVC and UVB bands. The second fluid filled flexible tube of the pair carries the emitted fluoresced light back to the converter 505 where it is delivered to the 300 to 4300 nm Raman Spectrometer 501 through a fiber optic link 504. A replaceable sterile sleeve 509 is placed over the distal end of the flexible fluid filled tube pair to allow reuse of the probe by multiple patients. The Raman Spectrometer extracts the spectra from the emitted light which is used to identify the infection microorganism and associated contamination level. A PC 508 is included to execute the AI DLNN locally which makes this embodiment a stand-alone system that executes the identification and characterization without an online connection. However, to retrieve the weights and biases of a specific pathogen, an online connection with the POCT server is required prior to executing the testing process.

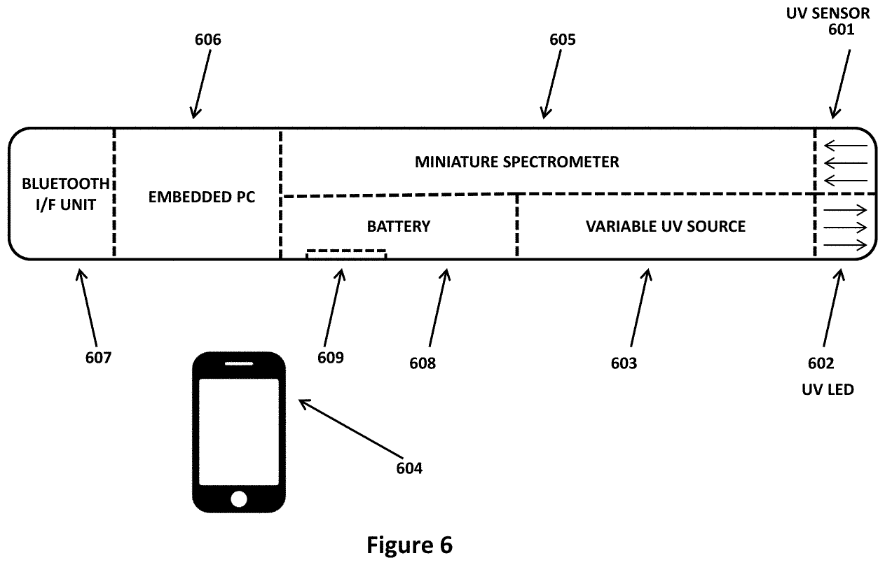

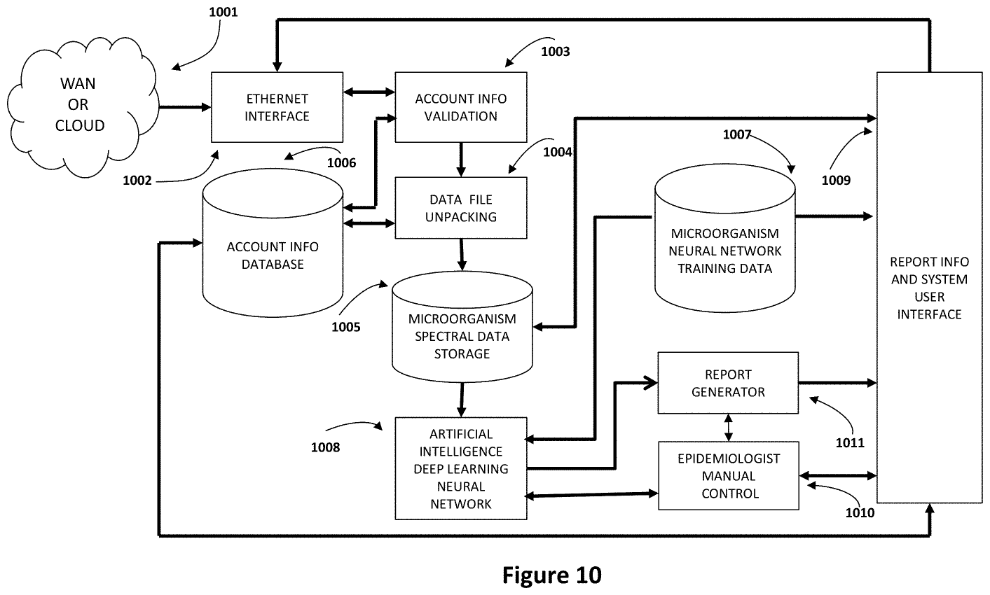

A third embodiment of the POCT invention is illustrated in FIG. 6. This configuration is for home or personal use and is a miniaturization of the first embodiment of the POCT unit. The physical configuration is a “Wand” (approx. 15 cm×2 cm×1 cm) device that is partially placed in the oral cavity 803, 811 during point-of-care-testing. The distal end of the POCT Wand contains a UV sensor 601 and a variable wavelength UV LED 602. The UV LED provides the fluorescence illumination source and the UV sensor receives the emitted light from the microorganism. A miniature spectrometer 605 is included to process the output of the UV sensor. The spectrometer data is formatted for delivery to the remote site AI application server farm (FIG. 10) by the embedded PC 606. A variable wavelength UV source generator 603 provides the selected wavelength determined by the control protocol software executed in the embedded PC 606. A Bluetooth interface unit 607 is included to link the Wand to a SmartPhone 604 which transfers the spectral data to the remote server site and displays the test results. The POCT Wand includes a battery 608 and associated charging interface connector 609.

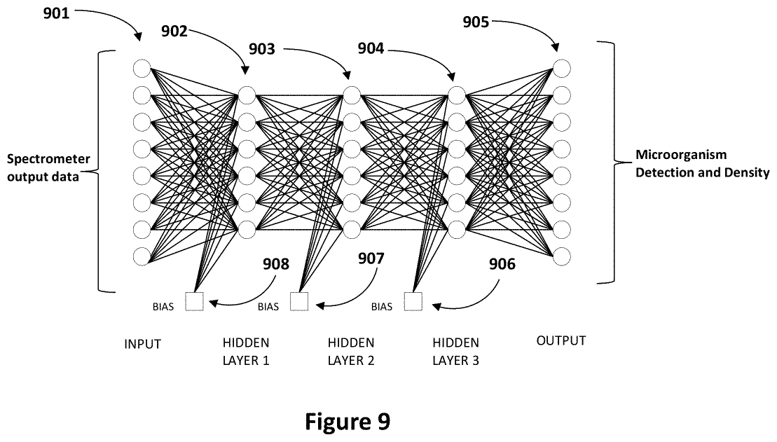

The basic neural network architecture used in this invention is illustrated in FIG. 9 and is provided as a shallow network for sake of explanation. The execution of the data set training and spectral pattern recognition is performed by high speed supercomputers at a remote site. The purpose of the AI DLNN is to identify the microorganism type and level of contamination. The neural network's input feature set 901 comes from the spectrometer output. This spectrometer data is the unique spectral patterns associated with the type and state of the microorganisms being tested. Each node input corresponds to a spectrum wavelength window energy level (i.e. wavelength, magnitude). An active microorganism has a unique spectral pattern based on its type and infection density.

The architecture of the neural network consist of hidden layers with multiple nodes 902, 903, 904 that are used to learn the weights and their associated biases 906, 907, 908. The hidden layers use a ReLu Activation Function and the output level uses a Sign Activation Function. The loss function is least squares regression whose results are used for the backpropagation process. The input level uses an Identity Activation Function.

This invention's Neural Network training data is derived from the spectral patterns of specific microorganisms in a laboratory environment. The Neural Network can be retrained for any microorganism spectral pattern of interest and therefore allows the creation of weighting and biases for multiple types of microorganisms. The particular target bacteria or virus is selectable by the clinician.

The training data set is created by collecting at least fifty samples of the subject pathogen at colony concentrations ranging from very low to very high. A spectral pattern measurement is done for each sample using the POCT UV Spectrometer. The spectral pattern data is used to train the DLNN using a backpropagation process to determine the weights and biases that best identify the subject pathogen. When using the POCT in the testing mode, the neural network output node indicates whether the subject pathogen is detected (i.e. positive or negative results). If the results is positive, an associated neural network the density level of the detected pathogen. The AI neural network process is equivalent to observing the microorganism microscopically and making a cognitive decision on the type and infection level of the microorganism. A detail description of this process is provided by A. Pandya and R. Macy^{[41]}.

The remote AI processing site block diagram is shown in FIG. 10. The POCT unit interfaces with the remote site through an internet link 1001. The link data is sent to an Ethernet interface 902 for distribution to the server farm database and application servers 1006, 1005, 1007, 1008. Patient account information is processed and sent to the account information database 1003, 1004. Account information is used to assist in tracking infected patients. The microorganism spectral data awaiting analysis resides in the microorganism spectral database 1005. The multimode multilevel DLNN is executed on very high speed supercomputers 1008 and the results sent to a report generator 1011. A clinician interface is provided for manual control 1010 and/or intervention in the testing process. A web site interface 1009 allows observation and interaction of the entire remote site process from a SmartPhone or PC.

LIST OF REFERENCE NUMERALS

| | |
|---|---|
| 101 | normal blood spectral pattern |
| 202 | blood infected with Denque Virus |
| 301 | Spectrum of living and dead E. coli |
| 401 | UV Spectrometer |
| 402 | Variable Wavelength Source |
| 403 | Fiber-optic cable |
| 404 | Fiber-optic cable |
| 405 | Fiber optic to fluid tube connector |
| 406 | Dual fluid filled tubes |
| 407 | Quartz tip lens |
| 408 | SmartPhone |
| 409 | Embedded PC |
| 410 | Sterile Sleeve |
| 501 | Spectrometer |
| 502 | Variable UV source |
| 503 | Fiber optic cable |
| 504 | Fiber optic cable |
| 505 | Fiber optic to fluid tube connector |
| 506 | Dual fluid filled tubes |
| 507 | Quartz tip lens |
| 508 | Lap-top PC |
| 509 | Sterile Sleeve |
| 601 | UV Sensor |
| 602 | UV LED |
| 603 | Variable UV Source |
| 604 | SmartPhone |
| 605 | Miniature Spectrometer |
| 606 | Embedded PC |
| 607 | Bluetooth Interface |
| 608 | Battery |
| 609 | Battery Charger Connector |
| 701 | Dual tube probe unit |
| 702 | Lips |
| 703 | Nasal Cavity |
| 704 | Palate |
| 705 | Oral Cavity |
| 706 | Pharynx |
| 707 | Epiglottis |
| 708 | Larynx opening into pharynx |
| 709 | Esophagus |
| 710 | Larynx |
| 801 | Point-of-Care-Test WAND |
| 802 | Lips |
| 803 | Nasal Cavity |
| 804 | Palate |
| 805 | Oral Cavity |
| 806 | Pharynx |
| 807 | Epiglottis |
| 808 | Larynx opening into pharynx |
| 809 | Esophagus |
| 810 | Larynx |
| 901 | Input Nodes |
| 902 | Hidden Layer 1 Nodes |
| 903 | Hidden Layer 2 Nodes |
| 904 | Hidden Layer 3 Nodes |
| 905 | Output Nodes |
| 906 | Layer 3 Bias |
| 907 | Layer 2 Bias |
| 908 | Layer 1 Bias |
| 1001 | WAN OR CLOUD |
| 1002 | Ethernet Interface |
| 1003 | Account Info Validation |
| 1004 | Data File Unpacking |
| 1005 | Microorganism Spectral Database |
| 1006 | Account Info Database |
| 1007 | Neural Network Training Data |
| 1008 | AI DLNN Application Processors |
| 1009 | Web Interface |
| 1010 | Manual Control Interface |
| 1011 | Report Generator |

We claim:

1. A programmable, mobile, and reusable point-of-care-testing device for identification and characterization of pathogens present in the respiratory airway; said device comprising:
A Raman Optical Spectrometer to detect the presence of a pathogen and programmable Deep Learning Neural Networks to analyze the spectral content of the emitted fluoresced light. The apparatus components include:
a optical spectrometer for obtaining the spectral content of the fluoresced emitted light;
a variable wavelength UV source that is used to fluoresce a pathogen at its highest energy state;
a fiber-optic cables that is a component of the testing probe apparatus;
a fiber-optic to fluid tube converter that is a component of the testing probe apparatus;
a fluid filled flexible tubing that is a component of the testing probe apparatus;
a quartz lens that is a component of the testing probe apparatus;
a quartz sleeve that allows reuse of the point-of-care-testing system;
a embedded processor that interfaces the spectrometer output data to a SmartPhone for delivery to a remote site for identification of the pathogen by the Deep Learning Neural Network;
a Deep Learning Neural Network Software that is reprogrammable to optimize identification of a particular pathogen;
a embedded processor that optimizes the illumination wavelength for optimal emitted energy;
a embedded processor that provides a Bluetooth or USB connection to a SmartPhone;
a remote application server that execute the Deep Learning Neural Network software; and
a pathogen spectral training set data obtained in a controlled laboratory environment.

2. The method of claim 1, wherein a personal computer is used at testing site to execute the Deep Learning Neural Network Software.

3. The method of claim 1, wherein the device components are miniaturized to fit into a wand configuration for insertion into the oral cavity.
The apparatus components include:
a miniature optical spectrometer for obtaining the spectral content of the fluoresced emitted light;
a variable wavelength LED UV source that is used to fluoresce a pathogen at its highest energy state;
a dual quartz lens that forms the optical interface for the illumination and emitted light apparatus;
a embedded processor that interfaces the spectrometer output data to a Bluetooth or USB to a SmartPhone or personal computer for delivery to a remote site for identification of the pathogen by the Deep Learning Neural Network;
a embedded processor that optimizes the illumination wavelength for optimal emitted energy;
a remote application server that execute the Deep Learning Neural Network software; and
a pathogen spectral training set data obtained in a controlled laboratory environment.

4. The method of claim 1, wherein training data for the associated Neural Network is derived in a laboratory environment for each specific pathogen type.

5. The method of claim 1, wherein the fluoresced microorganism's spectral pattern is used to detect its presence.

6. The method of claim 1, wherein the probe component is inserted in the oral cavity or nasal sinus of the patient.
